(19) United States
(12) Patent Application Publication
(10) Pub. No.: US 2004/0254395 A1
(43) Pub. Date: Dec. 16, 2004

(54) Ozonolysis process for preparing menthyl glyoxylate hydrate

(76) Inventors: Phillip Michael Cook, Kingsport, TN (US); Jerry Morris Keller, Dandridge, TN (US); Xiao Guang Yang, Tai Shui Wai (HK)

Correspondence Address: Michael J. Blake, Eastman Chemical Company, P.O. Box 511, Kingsport, TN, 37662-5075, US

(21) Appl. No.: 10/737,560

(22) Filed: Dec. 16, 2003

Related U.S. Application Data
(60) Provisional application No. 60/435,076, filed on Dec. 20, 2002.

(51) Int. Cl. C07C 69/66

(52) U.S. Cl. 560/174

(57) ABSTRACT

A process for producing menthyl glyoxylate hydrate (MGH), which comprises reacting a sodium or potassium salt of a monomenthyl maleate with a mixture of ozone and air in an aqueous reaction medium at a temperature of 15° to 50° C. to form MGH, wherein the reaction medium is circulated through a diffuser comprising a static in-line mixer or a packed column, and isolating the resulting MGH. The product is isolated from the reaction mixture, optionally purified by recrystallization, and dried. The ozone content in the ozone/air mixture is typically about 1% to about 5% by weight of the gas stream.

This application claims the benefit of U.S. Provisional Application Ser. No. 60/435,076, filed Dec. 20, 2002.

This invention relates to an improved process for obtaining menthyl glyoxylate hydrate (MGH) via a process that employs an ozonolysis reaction.

### BACKGROUND

Menthyl glyoxylate hydrate (MGH) is an important chiral building block in carbocyclic and heterocyclic chemistry. The menthyl alcohol portion contains three chiral centers and the aldehyde functionality readily undergoes a substantial number of reactions.

MGH has been synthesized by a variety of known chemical pathways.

For example, MGH may be prepared by reducing menthyl oxalyl chloride (from menthol and oxalyl chloride) with tri-n-butyltin hydride, as performed by Hub et al., J. Org Chem, Vol. 35, No. 11, page 3691 (1970). Subsequent preparations such as, for example, U.S. Pat. No. 5,442,094 to Schouteeten focused on more direct methods, wherein glyoxylic acid is esterified with menthol and then isolated and purified by conversion to a bisulfite complex. See also, Whitesell et al., J. Org. Chem. Vol. 35, No. 11, page 3691 (1970); and Fernandez et al., Synthetic Communications, 20(18), page 2837 (1990).

MGH has also previously been prepared via ozone reactions. For example, U.S. Pat. No. 5,015,760 to Sajtos describes preparation of alkyl glyoxylates by ozonolysis of dimenthyl maleate in organic solvents followed by catalytic hydrogenation. See also, PCT application WO 96/22960, which describes a process in which menthyl glyoxylate is prepared by ozonolysis of dimenthyl maleate.

U.S. Pat. No. 5,939,580 to Kloimstein et al. describes preparation of glyoxylate esters in which water-soluble salts of maleic or fumaric monoesters are subjected to ozonolysis in an aqueous solution without subsequent reduction of the ozonolysis reaction mixture. The laboratory scale procedure in U.S. Pat. No. 5,939,580 employs an ozone-containing oxygen stream; on a large- or plant-scale operation of that process, the attendant safety issues with handling large volumes of oxygen would be significant.

### BRIEF DESCRIPTION OF THE FIGURE

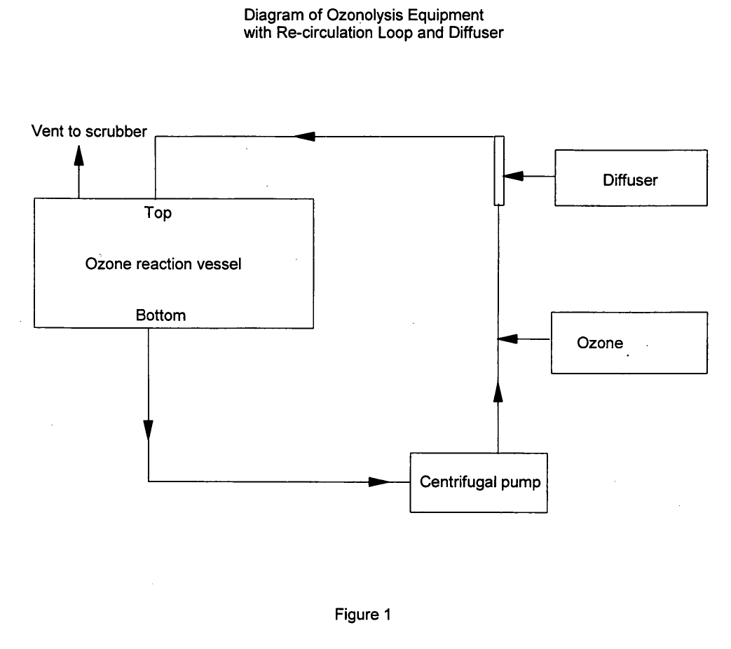

The accompanying FIG. 1 shows a typical equipment design for ozonolysis of MGH according to the present invention, which includes using a re-circulating loop containing a gas/liquid stream diffuser comprising an in-line static mixer or a packed column.

### SUMMARY

The present invention is a process for producing menthyl glyoxylate hydrate (MGH), which comprises reacting a sodium or potassium salt of a mono menthyl maleate with a mixture of ozone and air in an aqueous reaction medium at a temperature of about 15° to about 50° C. to form MGH, wherein the reaction medium is circulated through a diffuser comprising a static in-line mixer or a packed column, and isolating the resulting MGH. The ozone content in the ozone/air mixture is typically about 1 to about 5% by weight.

### DETAILED DESCRIPTION

The present invention is a process for producing menthyl glyoxylate hydrate (MGH), which comprises reacting a sodium or potassium salt of a monomenthyl maleate with a mixture of ozone and air in a well-agitated, or stirred, aqueous reaction medium at a temperature of about 15° to about 50° C. to form MGH. The reaction medium is continuously circulated through a diffuser comprising a static in-line mixer or a packed column, and the resulting MGH is then isolated. The ozone content in the ozone/air mixture is typically about 1 to about 5% by weight. The product is isolated from the reaction mixture, optionally purified by recrystallization, and dried.

The starting potassium or sodium salt of the monomenthyl maleate may be obtained by means known to those of skill in the art. See, for example, Annalen d. Chemie, 492, page 273 (1935); Chem. Ber., 119, page 3494 (1986); and the examples in U.S. Pat. No. 5,939,580. A process for making the salt of the sodium monomenthyl maleate, however, includes neutralizing a monomenthyl maleate with sodium hydroxide or sodium carbonate in a water solution. The monomenthyl maleate itself is prepared by reacting a stoichiometric amount of menthol with maleic anhydride in a molar ratio of 1:0.5. This can be carried out optionally in an inert solvent capable of forming an azeotrope with water, or the reaction can be carried out without a solvent. In either case, the reaction mixture is extracted with an aqueous alkaline sodium salt such as sodium hydroxide or sodium carbonate.

Ozone in the present invention is supplied to the reaction medium in the reaction zone by an ozone generator, in which compressed air is subjected to a high voltage electrical discharge. As the scale of the reaction is increased, however, safety issues arise relating to storage and handling of large volumes of oxygen. When using compressed air in the present process, however, such safety concerns are alleviated, but the reaction rate is reduced because the feed gas from the generator will necessarily contain much less ozone than if a pure oxygen feed were used, resulting in longer reaction times. The ozone content in the ozone/air mixture is typically about 1-5% by weight based on the total weight of the gas stream, and preferably should be about 2% or about 3% to about 5% by weight, and more preferably about 4% to about 5% by weight. It is to be understood that the foregoing lower or upper endpoints in the weight % range of ozone in the ozone/air mixture may be combined and sub-combined into any range falling within about 1% to about 5% by weight. The foregoing weight % figures correspond to a value of about 5 g to about 30 g of ozone per cubic meter of gas in the gas stream.

It has been discovered that the reaction time can be significantly reduced and product yield increased by use of a re-circulation loop having a static mixer or a packed column as a diffuser. The re-circulation through the diffuser serves to maximize the gas/liquid interface leading to saturation of the reaction medium with ozone. Moreover, this also allows the reaction with ozone to be performed with a safer, and easier to handle, compressed air carrier gas in fully acceptable space-time yields.

As reflected in FIG. 1, in addition to a reaction vessel and an ozone generator, the process of the present invention typically includes a re-circulation loop having an in-line static mixer or a packed column as a diffuser, and a centrifugal pump. The ozone reaction vessel may be a flask having various features apparent to those of skill in the art, including, for example, an external heating/cooling jacket, thermometer, stirrer and means for venting gas from the vessel. The reaction mixture in the reaction vessel or flask should also be continuously stirred while the ozonolysis is in progress. This serves to maintain dispersion of the precipitated MGH throughout the reaction mixture and eliminates build up on the wall.

While in principle the tube of the in-line mixer can be of virtually any length and diameter, its size is limited to the reactor volume being pumped through the tube, and the pump must be capable of overcoming the head pressure through that tube. Thus, the aspect ratio (length to inside diameter) of the in-line mixer's tube should be about 10 to 50, preferably 20 to 25. An aspect ratio of about 20 to 25 in the in-line mixer provides the best trade off between reaction yield, time, and pump head pressure. Within the tube of the in-line static mixer is typically one or more strips of non-reactive material, usually metal, such as one or more thin strips of stainless steel, or a similar structure meant to diffuse the reaction medium. Various types of static mixers are commercially available, depending on the design of the mixing element. The name “static” is due to the fact that they contain no moving parts, such as stirrers, and do not require a motor to create motion. Fluids entering a static mixer are typically divided by baffles, and mixing occurs by the continual splitting and recombining of flow streams. The continuous dividing of flows helps to ensure uniformity in composition, concentration, viscosity and temperature.

In addition, MGH yield can be increased and reaction time decreased by incorporating a packed column as the diffuser in the re-circulating loop. The column can be of various sizes and diameters. Column packing can be of various materials such as ceramic stones or saddles, or other ceramic materials, glass, metal, or plastic. It is preferred that the column packing be inert under the conditions of the reaction; that is, the packing should be substantially unreactive with the starting materials, the ozone, or the resulting MGH. It is also preferred that the size and shape of the packing be such that the surface area is maximized yet causes minimal interruption to flow. Glass or ceramic hollow spheres or tubes are preferred. The packing within the column typically should occupy greater than about 80% of the interior volume of the column. While in principle the packed column can be of virtually any length and diameter, the column size is limited to the reactor volume being pumped through the column. In addition, the pump must be capable of overcoming the head pressure through the column. Thus the aspect ratio (length:inside diameter) of the column should be about 10 to about 50, preferably about 20 to about 25.

The column is packed to maximize the mixing of the ozone-laden air stream with the aqueous solution of monomenthyl maleate salt. This insures maximum saturation of the reaction mixture with ozone. The column packing must be chosen to maximize the tortuous path traveled by the reaction mixture through the packed column yet not create a head pressure that can not be overcome by the pump.

While ozone can be introduced at virtually any point throughout the system, it is preferred to have the ozone/air mixture introduced immediately after the centrifugal pump in the pump-around circulation loop.

The ozonolysis reaction may be carried out within the temperature range of about 15° to about 50° C. The preferred temperature range is about 20° to about 25° C. A higher temperature increases reaction rate as well as the solubility of the product. The yield, however, is reduced because increased solubility of the product encourages further reaction with ozone. Lower temperature enhances precipitation of the product but also slows the reaction rate significantly, such that at about 15° C., limited or essentially no reaction occurs.

It has also been discovered that the pH of the reaction mixture should be maintained at from about 7.5 to about 9.5, preferably from about 8.5 to about 9.5, while the ozonolysis is in progress. If this is not done, the rate of formation of product slows substantially because acidic impurities such as oxalic and formic acids are formed as by-products during the ozonolysis. As the concentrations of such acidic impurities increases, the pH of the reaction mixture drops and leads to protonation of monomenthyl maleate carboxylate salt, which in turn causes precipitation of monomenthyl maleate. Once monomenthyl maleate is no longer dissolved, it becomes essentially protected from reaction with ozone. Various aqueous buffers known to those of skill in the art can be used to adjust the solution pH, such as: KH₂PO₄/borax (7-9.2), borax/HCl (7.6-8.9), KH₂PO₄/Na₂HPO₄ (7-7.5), carbonate or bicarbonate or mixtures thereof such as sodium carbonate/sodium bicarbonate, sodium carbonate/potassium bicarbonate, potassium carbonate/sodium bicarbonate, and potassium carbonate/potassium bicarbonate. Use of sodium carbonate/sodium bicarbonate mixtures is preferred for maintaining the pH in the range of about 8 to about 9.5

The use of an antifoaming agent is necessary since a significant amount of foaming occurs during the introduction of the air/ozone stream into the reaction mixture. Organic solvents such as isopropyl alcohol, 1-octanol, and 2-ethylhexanol can be used to control foaming but multiple aliquots must be added to the reaction mixture throughout the ozonolysis. Silicone-based antifoams are more efficient than alkanols. Silicone-based antifoams that can be used include, for example: Surfynol DF-37, DF-58, and DF-62, Wacker SRE and SE 21, Antifoam A emulsion from Fluka, and Antifoam agent 5701 from Kei Tat Chemicals, Ltd. The amount of antifoam agent used is 0.5% to 0.001% based on the overall weight of the reaction mixture. Antifoam A emulsion from Fluka is a preferred antifoaming agent because multiple additions are minimized and it appears to be the most resistant to ozone breakdown. Its use results in less silicon-based residuals in the final product compared to others, such as Wacker SRE. For commercial scale application, the antifoam may be continuously aspirated into the reaction mixture.

Filtration or centrifugation of the precipitated product may be used to isolate MGH from the reaction mixture. Extraction with an organic solvent is not useful, since impurities formed during the ozonolysis also are extracted and interfere with precipitation of MGH from the extracting solvent.

Further treatment of the crude MGH product is generally required to achieve a target level of >97% minimum assay by HPLC. The major impurities that contaminate the crude MGH include monomenthyl maleate sodium or potassium salt, oligomeric self-condensation products of MGH, and impurities formed by continued oxidation of the MGH product itself. Rinsing or re-slurring the crude product provides unsatisfactory results. On the other hand, recrystallization of the crude MGH product using hydrocarbon-based solvents such as hexane, heptane, cyclohexane, and branched aliphatic hydrocarbons containing up to and including 12 carbons provides satisfactory results. Heptane, hexane, and mixtures thereof are preferred.

The solvent-wet MGH is dried in a vacuum dryer at 25° to 35° C. The vacuum dryer may be a rotary dryer, such as a double cone vacuum dryer, or a stationary vacuum dryer, such as a vacuum tray dryer. The melting point of MGH is reported to be 83-85° C. However, if the drying temperature exceeds 35° C. during the early stage of the drying, the product may melt. The drying temperature may eventually be increased as the organic solvent evaporates.

By incorporating a re-circulation loop equipped with a diffuser, such as a static mixer or a packed column, the process provides improved yields of MGH. This can be viewed by comparison of Examples 14, Examples 5-7, and Examples 8-19 (excluding Example 14). The average yield of MGH obtained in Examples 14, which do not incorporate the use of a re-circulation loop containing a diffuser, is about 39% based on the dry weight of monomenthyl maleate salt. Incorporation of a re-circulation loop equipped with a static mixer as the diffuser, as in the present invention, provides an average yield of MGH of about 61% as described in Examples 5-7. Further, replacing the static mixer with a column packed with a variety of packing materials as described in Experiments 8-19 (excluding Example 14), the average yield of MGH is about 70%. The present examples show that incorporating a re-circulation loop, which has either a static mixer or packed column as a diffuser, significantly improves the yield of MGH as compared to a process run in the absence of such a diffuser.

This invention can be further illustrated by the following examples, although it will be understood that these examples are included merely for purposes of illustration and are not intended to limit the scope of the invention unless otherwise specifically indicated. The results of the following Examples are reported on Table. Ozone was generated in the following Examples using a Polymetrics Model T408 ozone generator.

### EXAMPLES 1-4

Preparation of MGH By Ozonolysis of Monomenthyl Maleate Sodium Salt in Aqueous Solution (Example 1) (Examples 1-4 show baseline ozonolysis technology in which a static in-line mixer or a column is not used.) Into a 2-L reaction vessel is introduced 388 g (0.17 moles) of 12.1% aqueous monomenthyl maleate sodium salt solution and 1.0 g of Wacker SRE antifoam. More antifoam is added as needed during the reaction period. The temperature of the reaction mixture is adjusted to 30°-35° C. and then subjected to ozone in a compressed air stream for 96 hours. At this point the concentration of monomenthyl maleate sodium salt is <2% by weight of the reaction mixture according to thin layer chromatography. During the reaction period, ozonator settings, pH (8.5-9.1 desired), temperature, and reaction progress are monitored. As the ozonolysis reaction progresses, the reaction mixture thickens and eventually MGH begins to precipitate. Water, lost by evaporation, during the ozonolysis, is replaced periodically during the reaction period. The pH is maintained in the desired range by addition of aqueous sodium carbonate solution as needed. When ozonolysis is determined to be complete by TLC, the reaction mixture is filtered and the filter cake is washed two times with 200 g of 5% aqueous solution of sodium bicarbonate. The assay of the crude MGH is 94% by HPLC. The water-wet MGH is dissolved in hexane (2 parts hexane by weight to 1 part of water-wet filter cake) at 60°-65° C. and extracted sequentially with 100 grams of 5% aqueous sodium bicarbonate and 100 grams of water. Water, in an amount equal to 10% by weight of the hexane, is added. The batch is agitated well to disperse the water and with continued agitation, cooled to 20°-25° C. At this temperature the product crystallizes and precipitates within 1-2 hours. The batch is then cooled to 0-10° C. and held at this temperature for 2-3 hours. The batch is filtered and the filter cake is washed twice with 50 g portions of cold hexane. The filter cake is then dried at 25-30° C. for 24 hours. The yield of MGH as a white powder is 17.6 g (40% based on monomenthyl maleate salt). The assay of the white powdered product by HPLC is 98 wt. %.

Examples 2, 3, and 4 were prepared similarly. (See Table.)

### EXAMPLES 5-7

Preparation of Menthyl Glyoxylate Hydrate By Ozonolysis of Monomenthyl Maleate Sodium Salt in Aqueous Solution (Example 5) (Examples 5-7 were prepared using a pump-around circulation loop containing a static in-line mixer).

Equipment such as described in FIG. 1 is used to prepare Examples 5, 6, and 7 according to the procedure given below. The diffuser is a static mixer, which is an 8-inch ribbon of stainless steel enclosed in glass tubing.

Into the 2-L reaction vessel is introduced 392 g (0.18 moles) of 12.7% aqueous monomenthyl maleate sodium salt solution and 1.0 g of Wacker SRE antifoam. More antifoam is added as needed during the reaction period. The temperature of the reaction mixture is adjusted to 30-35° C. and then subjected to ozone in a compressed air stream using a static mixer for 23 hours. At this point the concentration of monomenthyl maleate sodium salt is <2% by weight of the reaction mixture according to thin layer chromatography. During the reaction period, ozonator settings, pH (8.5-9.1 desired), temperature, and reaction progress are monitored. As the ozonolysis reaction progresses, the reaction mixture thickens and eventually MGH begins to precipitate. Water, lost by evaporation, during the ozonolysis, is replaced periodically during the reaction period. The pH is maintained in the desired range by addition of aqueous sodium carbonate solution as needed. When ozonolysis is determined to be complete by TLC, the reaction mixture is filtered and the filter cake is washed two times with 200 g of 5% aqueous solution of sodium bicarbonate. The assay of the crude MGH is 93% by HPLC. The water-wet MGH is dissolved in hexane (2 parts hexane by weight to 1 part of water-wet filter cake) at 60-65° C. and extracted sequentially with 100 grams of 5% aqueous sodium bicarbonate and 100 grams of water. Water, in an amount equal to 10% by weight of the hexane, is added. The batch is agitated well to disperse the water and with continued agitation, cooled to 20-25° C. At this temperature the product crystallizes and precipitates within 1-2 hours. The batch is then cooled to 0-10° C. and held at this temperature for 2-3 hours. The batch is filtered and the filter cake is washed twice with 50 g portions of cold hexane. The filter cake is then dried at 25-30° C. for 24 hours. The yield of MGH as a white powder is 28.8 g (62% based on monomenthyl maleate salt). The assay of the white powdered product by HPLC is 99 wt. %.

Examples 6 and 7 were prepared in similar manner, except the reaction temperature and reaction times are varied. (See Table.)

### EXAMPLES 8 & 9

Preparation of Menthyl Glyoxylate Hydrate By Ozonolysis of Monomenthyl Maleate Sodium Salt in Aqueous Solution (Example 9) (Examples 8 and 9 were prepared using a pump-around circulation loop containing a 30 cm glass column packed with 6×8 mm polytetrafluoroethylene tubes).

The equipment described in FIG. 1, using a packed column as the diffuser, is used to prepare Examples 8 and 9 according to the procedure given below.

Into the 2-L reaction vessel is introduced 395 g (0.17 moles) of 11.9% aqueous monomenthyl maleate sodium salt solution and 1.0 g of Wacker SRE antifoam. More antifoam is added as needed during the reaction period. The temperature of the reaction mixture is adjusted to 23-28° C. and then subjected to ozone in a compressed air stream for 7.6 hours. At this point the concentration of monomenthyl maleate sodium salt is <2% by weight of the reaction mixture according to thin layer chromatography. During the reaction period, ozonator settings, pH (9.0-9.5 desired), temperature, and reaction progress are monitored. As the ozonolysis reaction progresses, the reaction mixture thickens and eventually MGH begins to precipitate. Water, lost by evaporation, during the ozonolysis, is replaced periodically during the reaction period. The pH is maintained in the desired range by addition of aqueous sodium carbonate solution as needed. When ozonolysis is determined to be complete by TLC, the reaction mixture is filtered and the filter cake is washed two times with 200 g of 5% aqueous solution of sodium bicarbonate. The water-wet MGH is dissolved in hexane (2 parts hexane by weight to 1 part of water-wet filter cake) at 60-65° C.° C and extracted sequentially with 100 grams of 5% aqueous sodium bicarbonate and 100 grams of water. Water, in an amount equal to 10% by weight of the hexane, is added. The batch is agitated well to disperse the water and with continued agitation, cooled to 20-25° C. At this temperature the product crystallizes and precipitates within 1-2 hours. The batch is then cooled to 0-10° C. and held at this temperature for 2-3 hours. The batch is filtered and the filter cake is washed twice with 50 g portions of cold hexane. The filter cake is then dried at 25-30° C. for 24 hours. The yield of MGH as a white powder is 31.6 grams (72% based on monomenthyl maleate salt). The assay of the white powdered product by HPLC is 99 wt. %.

Example 8 was prepared similarly. (See Table.)

### EXAMPLES 10 & 11

Preparation of Menthyl Glyoxylate Hydrate By Ozonolysis of Monomenthyl Maleate Sodium Salt in Aqueous Solution (Example 11) (Examples 10 and 11 were prepared using a pump-around circulation loop containing a 47 cm glass column packed with 6×8 mm polytetrafluoroethylene tubes.)

Into the 2-L reaction vessel is introduced 388 g (0.17 moles) of 12.1% aqueous monomenthyl maleate sodium salt solution and 1.0 g of Wacker SRE antifoam. More antifoam is added as needed during the reaction period. The temperature of the reaction mixture is adjusted to 23 -28° C. and then subjected to ozone in a compressed air stream for 6.5 hours. At this point the concentration of monomenthyl maleate sodium salt is <2% by weight of the reaction mixture according to thin layer chromatography. During the reaction period, ozonator settings, pH (9.0-9.5 desired), temperature, and reaction progress are monitored. As the ozonolysis reaction progresses, the reaction mixture thickens and eventually MGH begins to precipitate. Water, lost by evaporation, during the ozonolysis, is replaced periodically during the reaction period. The pH is maintained in the desired range by addition of aqueous sodium carbonate solution as needed. When ozonolysis is determined to be complete by TLC, the reaction mixture is filtered and the filter cake is washed two times with 200 g of 5% aqueous solution of sodium bicarbonate. The water-wet MGH is dissolved in hexane (2 parts hexane by weight to 1 part of water-wet filter cake) at 60-65° C. and extracted sequentially with 100 grams of 5% aqueous sodium bicarbonate and 100 grams of water. Water, in an amount equal to 10% by weight of the hexane, is added. The batch is agitated well to disperse the water and with continued agitation, cooled to 20-25° C. At this temperature the product crystallizes and precipitates within 1-2 hours. The batch is then cooled to 0-10° C. and held at this temperature for 2-3 hours. The batch is filtered and the filter cake is washed twice with 50 g portions of cold hexane. The filter cake is then dried at 25-30° C. for 24 hours. The yield of MGH as a white powder is 35.6 g (81 % based on monomenthyl maleate salt). The assay of the white powdered product by HPLC is 99 wt. %.

Example 10 was prepared similarly. (See Table.)

### EXAMPLES 12-15

Preparation of Menthyl Glyoxylate Hydrate By Ozonolysis of Monomenthyl Maleate Sodium Salt in Aqueous Solution (Example 15) (Examples 12 through 15 were prepared using a pump-around circulation loop containing a 47 cm glass column packed with 2×4 mm glass tubes.)

Into the 2-L reaction vessel is introduced 447 g (0.22 moles) of 13.6% aqueous monomenthyl maleate sodium salt solution and 1.0 g of Antifoam A emulsion from Fluka. More antifoam is added as needed during the reaction period. The temperature of the reaction mixture is adjusted to 30-35° C. and then subjected to ozone in a compressed air stream for 7.3 hours. At this point the concentration of monomenthyl maleate sodium salt is <2% by weight of the reaction mixture according to thin layer chromatography. During the reaction period, ozonator settings, pH (9.0-9.5 desired), temperature, and reaction progress are monitored. As the ozonolysis reaction progresses, the reaction mixture thickens and eventually MGH begins to precipitate. Water, lost by evaporation, during the ozonolysis, is replaced periodically during the reaction period. The pH is maintained in the desired range by addition of aqueous sodium carbonate solution as needed. When ozonolysis is determined to be complete by TLC, the reaction mixture is filtered and the filter cake is washed two times with 200 g of 5% aqueous solution of sodium bicarbonate. The water-wet MGH is dissolved in hexane (2 parts hexane by weight to 1 part of water-wet filter cake) at 60-65° C. and extracted sequentially with 100 grams of 5% aqueous sodium bicarbonate and 100 grams of water. Water, in an amount equal to 10% by weight of the hexane, is added. The batch is agitated well to disperse the water and with continued agitation, cooled to 20-25° C. At this temperature the product crystallizes and precipitates within 1-2 hours. The batch is then cooled to 0-10° C. and held at this temperature for 2-3 hours. The batch is filtered and the filter cake is washed twice with 50 g portions of cold hexane. The filter cake is then dried at 25-30° C. for 24 hours. The yield of MGH as a white powder is 51.7 g (91% based on monomenthyl maleate salt). The assay of the white powdered product by HPLC is 99 wt. %.

Samples 13 and 15 were prepared in similar fashion. The experiment to prepare Sample 14 had to be discontinued since the reaction temperature (16-23° C.) caused the viscosity of the reaction mixture to increase which prolonged reaction time. (See Table.)

### EXAMPLES 16-19

Preparation of Menthyl Glyoxylate Hydrate By Ozonolysis of Monomenthyl Maleate Sodium Salt in Aqueous Solution (Example 16) (Example 16 through 19 were prepared using a pump-around circulation loop containing a 47 cm glass column packed with small ceramic stones. The stones are 5 mm to 20 mm in length/width and are ˜2 mm thick.)

Into the 2-L reaction vessel is introduced 472 g (0.21 moles) of 12.3% aqueous monomenthyl maleate sodium salt solution and 1.0 g of Antifoam A emulsion from Fluka. More antifoam is added as needed during the reaction period. The temperature of the reaction mixture is adjusted to 25-30° C. and then subjected to ozone in a compressed air stream for 10 hours. At this point the concentration of monomenthyl maleate sodium salt is <2% by weight of the reaction mixture according to thin layer chromatography. During the reaction period, ozonator settings, pH (9.0-9.5 desired), temperature, and reaction progress are monitored. As the ozonolysis reaction progresses, the reaction mixture thickens and eventually MGH begins to precipitate. Water, lost by evaporation, during the ozonolysis, is replaced periodically during the reaction period. The pH is maintained in the desired range by addition of aqueous sodium carbonate solution as needed. When ozonolysis is determined to be complete by TLC, the reaction mixture is filtered and the filter cake is washed two times with 200 g of 5% aqueous solution of sodium bicarbonate. The water-wet MGH is dissolved in hexane (2 parts hexane by weight to 1 part of water-wet filter cake) at 60-65° C. and extracted sequentially with 100 grams of 5% aqueous sodium bicarbonate and 100 grams of water. Water, in an amount equal to 10% by weight of the hexane, is added. The batch is agitated well to disperse the water and with continued agitation, cooled to 20-25° C. At this temperature the product crystallizes and precipitates within 1-2 hours. The batch is then cooled to 0-10° C. and held at this temperature for 2-3 hours. The batch is filtered and the filter cake is washed twice with 50 g portions of cold hexane. The filter cake is then dried at 25-30° C. for 24 hours. The yield of MGH as a white powder is 42.9 g (79% based on monomenthyl maleate salt). The assay of the white powdered product by HPLC is 98 wt. %.

Samples 17 through 19 were prepared similarly, except that the reaction time and reaction temperature are varied. (See Table.)

| | Wt % | | | | | | | | | | |
|---|---|---|---|---|---|---|---|---|---|---|---|
| | MMA in | | | Column | Column | Rxn | | Rxn | | Antifoam | Overall |
| | water | | Column | size, | packing | Temp., | | time | Antifoam | amount | Yield¹, |
| Example | solution | Mole | type | cm | material | ° C. | pH | Hrs | type | (g) | % |
| 1 | 12.1 | 0.17 | None | None | NA | 30-35 | 8.5-9.1 | 96 | Wacker | 1.6 | 40 |
| 2 | 13.6 | 0.18 | None | None | NA | 34-39 | 8.5-9.1 | 111 | Wacker | 2.1 | 29 |
| 3 | 12.3 | 0.08 | None | None | NA | 32-37 | 8.5-9.1 | 44 | Wacker | 1.0 | 44 |
| 4 | 12.3 | 0.08 | None | None | NA | 36-41 | 8.5-9.1 | 71 | Wacker | 1.0 | 41 |
| 5 | 12.7 | 0.18 | Static | None | NA | 30-35 | 8.5-9.1 | 23 | Wacker | 1.6 | 62 |
| | | | mixer | | | | | | | | |
| 6 | 11.7 | 0.18 | Static | None | NA | 28-33 | 8.5-9.1 | 18 | Wacker | 2.3 | 62 |
| | | | mixer | | | | | | | | |
| 7 | 11.6 | 0.17 | Static | None | NA | 25-30 | 8.5-9.1 | 12.5 | Wacker | 2.3 | 58 |
| | | | Mixer | | | | | | | | |
| 8 | 11.9 | 0.17 | Packed | 30 | PTFE | 23-28 | 9.0-9.5 | 5.5 | Wacker | 2.0 | 54 |
| | | | column | | tube | | | | | | |
| | | | | | 6 × 8 mm | | | | | | |
| 9 | 11.9 | 0.17 | Packed | 30 | PTFE | 23-28 | 9.0-9.5 | 7.6 | Wacker | 1.5 | 72 |
| | | | column | | tube | | | | | | |
| | | | | | 6 × 8 mm | | | | | | |
| 10 | 11.9 | 0.17 | Packed | 47 | PTFE | 23-28 | 9.0-9.5 | 8.0 | Wacker | 2.0 | 77 |
| | | | column | | tube | | | | | | |
| | | | | | 6 × 8 mm | | | | | | |
| 11 | 12.1 | 0.17 | Packed | 47 | PTFE | 23-28 | 9.0-9.5 | 6.5 | Wacker | 1.7 | 81 |
| | | | column | | tube | | | | | | |
| | | | | | 6 × 8 mm | | | | | | |
| 12 | 12.2 | 0.21 | Packed | 47 | Glass | 19-26 | 9.0-9.5 | 5.4 | Fluka | 1.2 | 80 |
| | | | column | | tube | | | | | | |
| | | | | | 2 × 4 mm | | | | | | |
| 13 | 13.6 | 0.31 | Packed | 47 | Glass | 20-25 | 9.0-9.5 | 8 | Fluka | 1.2 | 85 |
| | | | column | | tube | | | | | | |
| | | | | | 2 × 4 mm | | | | | | |
| 14 | 13.6 | 0.20 | Packed | 47 | Glass | 16-23 | 9.0-9.5 | 6.4 | Fluka | 1.2 | — |
| | | | column | | tube | | | | | | |
| | | | | | 2 × 4 mm | | | | | | |
| 15 | 13.6 | 0.22 | Packed | 47 | Glass | 20-25 | 9.0-9.5 | 7.3 | Fluka | 1.2 | 91 |
| | | | column | | tube | | | | | | |
| | | | | | 2 × 4 mm | | | | | | |
| 16 | 12.3 | 0.21 | Packed | 47 | Small | 25-30 | 9.0-9.5 | 10.0 | Fluka | 1.5 | 79 |
| | | | column | | stone | | | | | | |
| 17 | 12.3 | 0.21 | Packed | 47 | Small | 25-30 | 9.0-9.5 | 8.5 | Fluka | 1.3 | 81 |
| | | | column | | stone | | | | | | |
| 18 | 12.3 | 0.09 | Packed | 47 | Small | 25-30 | 9.0-9.5 | 3.0 | Fluka | 1.2 | 78 |
| | | | column | | stone | | | | | | |
| 19 | 12.3 | 0.21 | Packed | 47 | Small | 30-35 | 9.0-9.5 | 8.0 | Fluka | 1.2 | 76 |
| | | | column | | stone | | | | | | |

¹Note:
Yield of MGH is based on the dry weight of starting material (monomenthyl maleate salt)

In the drawings and specification, there have been disclosed typical preferred embodiments of the invention and, although specific terms are employed, they are used in a generic and descriptive sense only and not for purposes of limitation, the scope of the invention being set forth in the following claims.

We claim:

1. A process for producing menthyl glyoxylate hydrate (MGH), which comprises reacting a sodium or potassium salt of a monomenthyl maleate with a mixture of ozone and air in a stirred aqueous reaction medium at a temperature of 15° to 50° C. to form MGH, wherein the mixture has an ozone content of about 1% to about 5% based on the total weight of the gas mixture and wherein the reaction medium is circulated through a diffuser comprising a static in-line mixer or a packed column.

2. A process according to claim 1 wherein the process further comprises the steps of isolating the MGH and purifying the MGH by recrystallizing the MGH.

3. A process according to claim 2 wherein the process further comprises isolating the purified MGH by filtration or centrifugation, and drying the resulting MGH product.

4. A process according to claim 1 wherein the temperature is about 20° to about 25° C.

5. A process according to claim 4 wherein the reaction medium is circulated through a packed column.

6. A process according to claim 5 wherein the aspect ratio of the column is about 20 to about 25.

7. A process according to claim 5 wherein the column is packed with beads or particles comprising glass, ceramic, metal, plastic or a mixture thereof.

8. A process according to claim 4 wherein the reaction medium is circulated through a static in-line mixer having an aspect ratio of about 20 to about 25.

9. A process according to claim 5 wherein the pH of the reaction mixture is maintained at a pH of about 7.5 to about 9.5.

10. A process according to claim 9, wherein the pH is maintained by adding to the reaction mixture an aqueous buffer selected from a group consisting of KH₂PO₄/borax, borax/HCl, KH₂PO₄/Na₂HPO₄, and mixtures of carbonates and bicarbonates.

11. A process according to claim 1 wherein the process additionally comprises recrystallizing the MGH in an organic solvent selected from the group consisting of hexane, heptane, cyclohexane, and linear or branched-chain aliphatic hydrocarbons having up to 12 carbons.

12. A process according to claim 11 wherein the process additionally comprises drying the recrystallized MGH.

13. A process according to claim 1 wherein the reaction medium further comprises an antifoaming agent selected from the group consisting of Surfynol DF-37, Surfynol DF-58, Surfynol DF-62, Wacker SRE, Wacker SE 21, Fluka Antifoam A emulsion, and Antifoam agent 5701.

14. A process according to claim 5 wherein the column is packed with glass or ceramic hollow spheres or tubes.

15. A process for producing menthyl glyoxylate hydrate (MGH), which comprises contacting a continuously stirred aqueous reaction medium comprising an antifoaming agent, a buffer, and a sodium or potassium salt of a monomenthyl maleate with a mixture of ozone and air at a temperature of 20° to 25° C. to form MGH, wherein the mixture has an ozone content of about 1% to about 5% based on the total weight of the gas mixture and wherein the reaction medium is circulated through a diffuser comprising a packed column.

16. A process according to claim 15, wherein the buffer is sodium carbonate or sodium bicarbonate or a mixture thereof, and the antifoaming agent is selected from the group consisting of Wacker SRE, Wacker SE 21, and Fluka Antifoam A emulsion.

17. A process according to claim 16 wherein the column is packed with glass or ceramic hollow spheres or tubes.

18. A process according to claim 15 wherein the process further comprises the steps of purifying the MGH by recrystallization and drying the purified MGH.
